(19) United States
(12) Patent Application Publication
(10) Pub. No.: US 2024/0189210 A1
(43) Pub. Date: Jun. 13, 2024

(54) USE OF A CATIONIC POLYMER FOR REINFECTION PREVENTION

(71) Applicant: Conopco, Inc., d/b/a UNILEVER, Englewood Cliffs, NJ (US)

(72) Inventors: Vidula IYER, Bangalore (IN); Jasmeet Kaur KHOKHAR, Delhi (IN); Sayandip MUKHERJEE, Bangalore (IN); Ajay Singh NEGI, Bangalore (IN); Bharath PALANISAMY, Bangalore (IN); Sandip Bhanudas PATHAK, Bangalore (IN)

(21) Appl. No.: 18/286,771

(22) PCT Filed: Apr. 20, 2022
(86) PCT No.: PCT/EP2022/060423

§ 371 Date: Oct. 13, 2023

(30) Foreign Application Priority Data
Apr. 21, 2021 (IN) 202121018489
Jun. 10, 2021 (EP) 21178796.5

(51) Int. Cl. A61K 8/81; A01N 43/36; A01P 1/00; A61K 8/02; A61K 8/06; A61Q 17/00; A61Q 19/10

(52) U.S. Cl. CPC A61K 8/817; A01N 43/36; A01P 1/00; A61K 8/0229; A61K 8/064; A61Q 17/005; A61Q 19/10

(57) ABSTRACT

The present invention relates to treating a surface with a polymer and compositions comprising it for preventing microbial reinfection especially that of virus and bacteria on the surface for a long period of time after the surface has been treated. The present invention also relates to certain liquid cleansing compositions, sanitizers, hand-held spray devices and towelettes comprising the polymer for delivering the above benefit.

### FIELD OF THE INVENTION

The present invention relates to treating a surface with a polymer and compositions comprising it for preventing microbial reinfection especially that of virus and bacteria on the surface for a long period of time after the surface has been treated. The present invention also relates to certain liquid cleansing compositions, devices and towelettes comprising the polymer.

### BACKGROUND OF THE INVENTION

Respiratory infections are considered to be one of the most prevalent cause of disease, worldwide. Globally, acute lower respiratory infections are an important cause of morbidity and mortality in children below 5 years of age. Scientific studies have identified respiratory syncytial virus (RSV), as the most common viral cause of death due to such infection; other prominent viruses being human metapneumovirus, parainfluenza viruses, influenza viruses A and B, adenoviruses and of recent origin coronavirus. Several tens of millions of such infections are reported every year leading to more than half a million deaths in children below the age of five, largely in low and middle income households.

Other than viral infections, bacterial infections also account for a large percentage of morbidity and mortality in the world. Bacteria found on the skin can be divided into two groups: resident and transient bacteria. Resident bacteria are Gram positive bacteria which are established as permanent microcolonies on the surface and outermost layers of the skin and play an important, role in preventing the colonization of other, more harmful bacteria and fungi. Transient bacteria are bacteria which are not part of the normal resident flora of the skin but can be deposited when airborne contaminated material lands on the skin or when contaminated material is brought into physical contact with it. Transient bacteria are also typically divided into Gram positive and Gram negative subclasses. Gram positive bacteria include pathogens such as Staphylococcus aureus, Streptococcus pyogenes and Clostridium botulinum. Gram negative bacteria include pathogens such as Salmonella, Escherichia coli, Klebsiella, Haemophilus, Pseudomonas aeruginosa, Proteus and Shigella dysenteriae. Gram negative bacteria are distinguished from Gram positive by an additional protective cell membrane which generally results in the Gram-negative bacteria being less susceptible to topical antibacterial actives.

Research has shown that virus and bacteria as discussed above, can transmit through touching by infected hands and even through inanimate surfaces including mobile phones. As a consequence, hand hygiene is a commonly recommended method of killing such germs and thereby reducing the risk of not only respiratory infection but also gastrointestinal diseases. Hand hygiene includes use of soap and running water to wash the hands which makes it not only free of dirt but also in washing away and killing germs including bacteria and virus. Alternately, alcohol-based hand sanitizers which contain very little water may be rubbed on the hands as another effective means of removing virus and bacteria from hands. While such methods are very good in instantaneously killing the germs, they do not provide long lasting post wash protection from germs which may invade the surface for up to several hours after the surface has been cleaned. Long lasting protection from germs would require an additional step of applying a leave-on composition on to the surface.

Thus, a cleansing composition comprising an antimicrobial active that not only kills germs but also deposits actives in sufficient quantities on the skin to ensure effective kill for many hours after the washing action is much desired by consumers.

The present inventors when looking for such actives hit upon a certain class of polymers which are cationic in nature and have a specific high charge density which not only kill germs like bacteria and virus but also ensure that for several hours post such a treatment, they prevent reinfection. Polydiallyldimethylammonium chloride (PDADMAC, also known as polyquaternium-6) is a cationic homopolymer of diallyldimethylammonium chloride belonging to the class of quaternary ammonium compounds is an example of such a polymer that the present inventors found to be effective as per the present invention.

Cationic polymeric compounds are known to have antimicrobial efficacy. The main positively charged moieties in these natural or synthetic structures are quaternary ammonium groups.

US2014/0294749 (Medivators Inc) discloses antimicrobial compositions, methods of preparing antimicrobial compositions, methods of using the antimicrobial compositions, and/or kits that include the antimicrobial compositions where ammonium chloride cationic species is used to effect the antimicrobial activity.

The mechanism of action of QAC's (quaternary ammonium compounds) against bacteria and yeast is well established. The mechanism of action of cationic agents against animal viruses have only recently been investigated. To the knowledge of the present inventors, such high charge density cationic polymers are not known for prevention of reinfection by microbes like virus and bacteria thus providing long lasting hygiene benefit and extended germ protection.

It is thus an object of the present invention to provide for prevention of reinfection by germs from surfaces or at least minimization of such reinfection.

It is another object of the present invention to provide for such benefit from a wash off, i.e. a cleansing composition.

### SUMMARY OF THE INVENTION

The present invention relates to use of a cationic polymer having a charge density higher than 2.0 meq/g for reinfection prevention against germs.

The germs, as per this invention, against which such reinfection is prevented are preferably virus and bacteria.

Another aspect of the present invention relates to a liquid cleansing composition comprising (a) a cationic polymer having a charge density higher than 2.0 meq/g (b) a surfactant and (c) higher than 65% water.Another aspect of the present invention relates to a method of minimizing regrowth of germs on a surface for up to 6 hours post cleaning comprising the step of cleaning the surface with a composition comprising a cationic polymer having a charge density higher than 2.0 meq/g.

### DETAILED DESCRIPTION OF THE INVENTION

These and other aspects, features and advantages will become apparent to those of ordinary skill in the art from a reading of the following detailed description and the appended claims.

For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated and may be abbreviated as ‘wt %’. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The present invention may be used to prevent reinfection by virus and bacteria on any type of surface. The surface may be inanimate or animate. Inanimate surfaces include surfaces in the home or outside. Inanimate surfaces whether at home or outside are generally classified as soft surfaces or hard surfaces. Soft surfaces include clothes worn by persons, upholstery, or those in the bedroom or living rooms like bedsheets, pillows or in the living room like curtains, sofas etc. Hard surfaces include those of metal, wood, porcelain, plastic, glass whether inside homes, or outside in common areas of buildings. Public areas like restaurants, bars and pubs, parks, bus stops, train stations and airplane terminals also have a large number of hard and soft surfaces where germs like virus and bacteria can reside and multiply at the right conditions.

Animate surfaces include the external surfaces of living organisms like plants and animals including humans. Due to the rich availability of nutrients and water on most animate surfaces, germs reside and are capable of multiplying on such surfaces. The present invention is used to prevent virus reinfection on any type of surface preferably on animate surfaces. The invention is especially useful for reinfection prevention on topical surface of the human body. The topical surfaces include skin on any external part of the body including scalp and hair. It also includes surfaces in the oral cavity. The present invention is especially useful for use on the external skin surface of the human body. The present invention may be used through a leave-on composition or a wash-off composition. By a leave-on composition is preferably meant that a composition which is applied on the external surface of the human body and allowed to remain thereon till the person takes a shower or a bath usually after several hours or after a day. By a wash-off composition is preferably meant that the composition is used to wash the external surface of the body e.g with a soap composition, a body wash, a face wash, a shampoo or a hair conditioning composition with copious amount of water such that substantial amount of dirt and oils are washed off the surface of the body leaving it clean. Such wash off compositions generally contain sufficient amounts of surfactants that enable the wash-off action. The present invention is especially suitable for delivery through a wash-off composition.

The cationic polymers for use in the present invention have a charge density higher than 2.0 meq/g. The charge density of the polymers may be measured using the following procedure:

The procedure involves use of a titration method. A positive colloid ion is titrated directly with a negative colloid ion. Normality of the titer solution is defined by number of equivalents of the dissociable groups of the polymer in one liter of solution, which may be determined by colloid titration using the known standard reagent or by direct or indirect estimations of the electrolytic groups by chemical analysis. The concentration of the titer solution is adequate between 0.001 N and 0.0001 N. The end point of the colloid titration is generally determined by color change of an indicator substance, but at some time when the metachromasia is more or less undecided, with the help of the sudden precipitation of the reactants. The blank titrations must be carried out. The pH of the titration medium is controlled with the addition of HCl or NaOH solution. The difference between the two titers at the same pH value is taken, and from it we can calculate the gram equivalents or number of equivalents per unit gram of the test material.

E.g. 5 ml. of 0.017% solution of chitosan hydrochloride (A) is placed in a small Erlenmeyer flask (or a large test tube) and an adequate amount of hydrochloric acid or sodium hydroxide is added (0.001 to 1 N, 0.1 to 1 ml.) to make the titration medium acidic or alkaline. Then one drop of 0.1% toluidine blue is added as an indicator. After shaking a few seconds, 0.000541 N potassium salt of polyvinyl sulfate (P.V.S K) solution is added titrimetrically from a semimicroburette or microburette until the blue color of the indicator changes into reddish purple; at the same time flocculation or precipitation of the reacting complexes appears abruptly. The hydrogen-ion concentration of each reaction medium is examined at the end of each titration. It is desirable for exact studies to use a glass electrode apparatus to measure the pH, but the use of the test papers is convenient for practical purposes. In the present invention, commercial test papers were generally used, the accuracy of which is ca. pH 0.1-0.3. As a blank titration, 5 ml. of distilled water was used in place of the chitosan hydrochloride solution; the other procedures being the same as above. The difference between both titrations at the same pH are taken. The negative polymer ions (P.V.S.-K) combining capacity per unit weight of chitosan hydrochloride is calculated from the following equation:

$$\frac{Equivalent}{gram} = \frac{0.000541 * Δ * 10 - 3 }{0.0170 * 5 * 10 - 2}$$

where Δ is the difference between the titers of the test and the control at the same pH value. The fluctuation of each titer lies below 0.03 ml. if carefully operated.

Polymers that may be used to deliver the benefits of the present invention include poly diallyl dimethyl ammonium chloride (PDADMAC), poly N-[3-(dimethylamino)propyl]methacrylamide (PDMAPMA), poly[2 (Dimethylamino)ethyl methacrylate] (PDMAEMA), polyethylene imine (PEI), chitosan, polyquaterium-16, polyquaternium-7, or polyquaternium-37, preferably PDADMAC. The charge density of the above mentioned materials as follows:

6.2 meq/g for PDADMAC, 5.6 meq/g for PDMAPMA, 4.2 meq/g for PDMAEMA, 4.05 meq/g for PEI, 3.7 meq/g for chitosan, 3.3 meq/g for polyquaternium-16, 3.1 meq/g for polyquaternium-7 and 2.3 meq/g for polyquaternium-37.

The preferred PDADMAC molecular weight for use in this invention is in the range of 2,00,000 to 20,00,000, preferably 4,00,000 to 6,00,000. It is a high charge density cationic polymer with a viscosity in the range of 10,000 to 20,000 mPa·s. The polymer is available under the trade name of Merquat-100 (INCI: Polyquaternium-6 or PQ-6) from Lubrizol Inc.

The invention is especially useful in providing protection against reinfection by a virus and bacteria long after the surface has been cleaned with a cleansing composition comprising the select cationic polymer having a charge density higher than 2.0 meq/g, preferably higher than 3.0 meq/g, further more preferably higher than 3.5 meq/g. Preferably the cationic polymer has a charge density of up to 8 preferably up to 7 meq/g. The reinfection prevention is seen to be effective for as much as 6 hours after the cleansing step and in some cases as much as 9 hours and in certain other cases up to 12 hours after the cleansing step. Thus, the invention provides for long lasting hygiene benefit or extended germ protection.

The cationic polymers used in the present invention preferably are homo polymer/copolymer having at least one nitrogen atom as part of its repeating unit. The nitrogen is so present as to include a % nitrogen of higher than 5%. Preferably nitrogen is present in the range of 5 to 40 wt % of the cationic polymer; more preferably 6 to 35 wt %, and most preferably 6 to 20 wt %.

Weight percent of nitrogen is calculated using the following equation:

Weight % of N=(Weight of N in repeating unit/total molecular weight of repeating unit)*100

Hence weight percent of nitrogen does not vary with degree of polymerisation/molecular weight of polymer.

The weight percent nitrogen of cationic polymers used in the present invention are: PQ-6 (8.7%), PQ-37 (7.2%) and PQ-16 (16%). Cationic polymers outside the range claimed in the present invention include PQ-10 (1.5 to 2.2% N) and PQ-67 (1.3% N).

### Application in Personal Care

The benefits of the invention may be delivered on to the topical surface of the body, scalp or hair though any cleansing composition produced for such use like a soap bar or a liquid hand wash composition (generally comprising a soap), a liquid/gel body wash or a shampoo composition (generally comprising a synthetic surfactant). Soaps and synthetic surfactants in wash compositions including soap are anionic in nature. While the present invention may be enabled by delivering the cationic polymer through any such wash off composition, it is especially preferred that the cationic polymer is delivered from such anionic surfactant containing composition where the cationic polymer is kept separated from the anionic surfactant till such time the consumer uses the composition. This is necessary since the two materials could undergo reaction with charge neutralisation leading to precipitation and making both the anionic and the cationic moiety ineffective. Any encapsulation technology that keeps the cationic polymer encapsulated in the wash off composition could be effective. An especially effective method of encapsulating the cationic polymer is by delivering it though a water-in-oil emulsion.

Thus, a preferred aspect of the present invention relates to use of a water-in-oil emulsion composition comprising (a) an aqueous phase comprising a cationic polymer having a charge density higher than 2.0 meq/g (b) a hydrophobic phase chosen from petrolatum or wax and (c) an emulsifier, for reinfection prevention against germs. Preferably, the use is non-therapeutic or cometic.

The cationic polymer meeting the charge density criterion is preferably included in a water-in-oil emulsion composition at 5 to 50%, more preferably 10 to 40%, by weight of the emulsion composition. The hydrophobic phase is chosen from petrolatum or wax, preferably petrolatum. The hydrophobic phase may also comprise smaller amounts of other hydrophobic materials like fatty acids, triglycerides, or silicones.

Petrolatum which is known as petroleum jelly is a purified mixture of semi-solid hydrocarbons obtained from petroleum with a carbon chain length of 25 or higher. The petroleum jelly has excellent moisturizing property and has a melting point ranging from a little below to a few degrees above 37° C. It is colorless or pale yellow (when not highly distilled), translucent and devoid of taste and smell when pure. It is insoluble in water. Preferred petrolatum for use in the present invention is one having a slip melting point in the range of 45 to 75° C. The hydrophobic phase is preferably included in 20 to 60%, more preferably 20 to 50% by weight of the water-in-oil emulsion composition.

“Wax” as used herein refers to a class of organic compounds that characteristically comprise long alkyl chains. Typically, the waxes are plastic (malleable) at about 25° C. “Wax ester” as used herein means ester which is comprised by a wax. The wax preferably has a melting point from 40° C. to 200° C., more preferably from 50° C. to 120° C.

The wax for use in the present invention is preferably suitable for use in a cosmetic composition. The wax may be natural wax and/or synthetic wax. Such waxes are often selected from hydrocarbon waxes and ester waxes but the wax preferably comprises wax ester. In some preferred embodiments, the wax comprises beeswax, rice bran wax, montan wax, spermaceti wax, carnauba wax, candelilla wax, sugarcane wax, insect wax, or a mixture thereof. More preferably, the wax comprises beeswax, rice bran wax, montan wax, carnauba wax, or a mixture thereof. Even more preferably, the wax is beeswax.

The emulsifier for use in emulsifying the water in the oil and keeping it in a stable condition is preferably a non-ionic surfactant. Preferred surfactants for use in the emulsion composition of the invention have an HLB value of less than 10, preferably between 2 and 7.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various non-ionic surfactants are given below:
A value <10: Lipid soluble (water insoluble)
A value >10: Water soluble
A value from 4 to 8 indicates an anti-foaming agent
A value from 7 to 11 indicates a W/O (water in oil) emulsifier
A value from 12 to 16 indicates oil in water emulsion
A value from 11 to 14 indicates a wetting agent
A value from 12 to 15 is typical of detergents
A value of 16 to 20 indicates a solubiliser or hydrotrope.

Suitable emulsifiers for use in the water-in-oil emulsion compositions of the invention are selected from sorbitan monostearate, sorbitan monooleate or combinations thereof. Sorbitan monostearate is sold under the brand name SPAN-60 and sorbitan monooleate is sold under the brand name SPAN-80. It is preferred that the emulsion is prepared using both of these emulsifiers. The emulsifier is included in 2 to 10%, preferably 2 to 8% by weight of the emulsion composition.

Water or the aqueous phase forms the dispersed phase in the water-in-oil emulsion. It is preferably present in 40 to 60% preferably 40 to 55% by weight of the emulsion.

The water-in-oil emulsifier is preferably prepared using the following process to ensure maximal inclusion of the cationic polymer in the water/aqueous phase. The steps comprise (a) mixing the emulsifier with the hydrophobic phase at a temperature in the range of 20 to 95° C.; followed by (b) mixing a solution/dispersion of a water-soluble skin benefit agent in water maintained at a temperature of 20 to 95° C., to the mixture of step (a). The preferred temperatures for both step (a) and step (b) are in the range of 70 to 85° C.

Another aspect of the present invention relates to use of a cleansing composition comprising the water-in-oil emulsion composition, as described above, additionally comprising an anionic surfactant preferably soap for reinfection prevention against germs. Preferably, the use is non-therapeutic or cosmetic.

By ‘a cleansing composition’ as used herein, is meant to include a composition for topical application to skin, hair and/or scalp of mammals, especially humans. Such a composition is generally applied on to the desired topical surface of the body for a period of time from a few seconds to up to a few minutes generally after diluting with water. After this period of time of application the composition is generally rinsed off with water or wiped away. It includes any product applied to a human body for also improving appearance, odor control or general aesthetics. The composition of the present invention can be in the form of a liquid, lotion, cream, foam, scrub, gel, shampoo, conditioner, handwash, facewash or bodywash product.

The cleansing composition could be in any format but is preferably a soap bar.

The cosmetically acceptable base comprises water in addition to the anionic surfactant. A particularly preferred anionic surfactant is soap. The water-in-oil emulsion composition is preferably included in 1 to 20%, preferably 2 to 10% by weight of the cleansing composition.

Thus, in effect, the cationic polymer is included in any composition for use in cleansing by way of the present invention in 0.05 to 10%, preferably 0.1 to 5%, more preferably 0.1 to 2% by weight of the cleansing composition. The cleaning is especially effective on topical surfaces of an animal or human.

The soap for preparing the cleansing composition of the invention is preferably a C₈-C₂₄ soap, more preferably C₁₀-C₂₀ soap and most preferably C₁₂-C₁₈ soap. The cation of the soap can be alkali metal, alkaline earth metal or ammonium. Preferably, the cation of the soap is selected from sodium, potassium or ammonium. More preferably the cation of the soap is sodium or potassium. Fatty acids derived from other suitable oils/fats such as groundnut, soybean, tallow, palm, palm kernel, etc. may also be used in other desired proportions.

A cosmetically acceptable base comprising the anionic surfactant forms the rest of the composition other than the water-in-oil emulsion present in the cleansing composition. Thus, the cosmetically acceptable base generally forms 80 to 99% by weight of the cleansing composition.

When present, the anionic surfactant e.g. soap, is preferably present in an amount of 1 to 90%, preferably from 10 to 85%, more preferably 25 to 75% by weight of the cleansing composition. The cleansing composition is preferably in the form of a solid or semi solid form, most preferably in a solid form. Preferred solid compositions are in the shape of a soap bar.

Other anionic surfactants are preferably selected from alkyl ether sulphate, primary alkyl sulphate, secondary alkyl sulphonates, alkyl benzene sulphonates, or ethoxylated alkyl sulphates. The anionic surfactant other than soap which is preferred in the cleansing composition is an alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C₆ and C₁₆.

Preferred cleansing compositions may include other known ingredients such as perfumes, pigments, preservatives, emollients, sunscreens, gelling agents and thickening agents. Choice of these ingredients will largely depend on the format of the composition.

Water is a preferred carrier. When water is present, it is preferably present in at least 1%, more preferably at least 2%, further more preferably at least 5% by weight of the composition. When water is the carrier, a preferred cleansing composition comprises 10 to 50%, more preferably 12 to 40%, further more preferably from 12 to 25% by weight water.

The cleansing composition of the invention may also be delivered through a moisturizing bar or a moisturizing liquid composition. Moisturizing bar compositions comprising fatty acyl isethionates (e.g. cocyl isethionate) are especially preferred.

Fatty acyl isethionates (e.g., cocoyl isethionates) surfactant “products” are defined as mixtures of anionic acyl isethionate surfactants and fatty acids/fatty acid soaps. They are highly desirable in personal care skin or hair cleansing products, particularly in personal care products, because they lather well, are mild to the skin and have good emollient properties. Typically, fatty acid isethionate surfactant products are produced by esterification of fatty acids or by reaction of fatty acid chloride having carbon chain length of C8 to C20 with isethionate. A typical surfactant product containing fatty acyl isethionate contains about 40 to 95 wt. % acid isethionate, and 5 to 50 wt. %, typically 10 to 40 wt. % free fatty acid, in addition to isethionate salts, typically at less than 5%, and trace (less than 2 wt. %) of other additives. Fatty acid soap may be included in the range of 5 to 15 wt %. Other surfactants like betaines may be included in 1 to 5 wt %. Water is generally included in 2 to 8 wt % of the composition.

Another aspect of the present invention relates to use of a soap bar composition comprising (a) 40 to 80 wt % soap and (b) a composite particle comprising a porous clay particle having a water holding capacity in the range of 10 to 50% by weight, coated with a cationic polymer having a charge density higher than 2.0 meq/g, and further coated there upon with a hydrophobic material selected from wax or petrolatum, for reinfection prevention against germs. Preferably, the use is non-therapeutic or cosmetic.

In this aspect of the present invention the wax or petrolatum as disclosed hereinabove for use in the water-in-oil emulsion composition may be used. The other ingredients of the soap bar like the soap itself are preferably those as disclosed hereinabove.

In this aspect of the invention where the soap composition comprises the composite particle, the cationic polymer is included in and/or coated on a porous clay particle. The clay is preferably one of a smectite class. Preferred clays are kaolin, bentonite or china clay. Clays are finely ground natural rock or soil material. Based on the physical and chemical nature these clays are further classified into many clay mineral groups. For example, the structure of kaolinite is a tetrahedral silica sheet alternating with an octahedral alumina sheet. The charges within the structural unit are balanced. The molecular formula that is common for the kaolinite group is Al₂Si₂O₅(OH)₄.

Bentonite has an ability to form thixotropic gels with water, an ability to absorb large quantities of water with an accompanying increase in volume of as much as 12-15 times its dry bulk, and a high cation exchange capacity. These cations are exchangeable due to their loose binding and, together with broken bonds (approximately 20% of exchange capacity), give montmorillonite a rather high (about 100 meq/100 g) cation exchange capacity, which is little affected by particle size. These clays could be procured from companies like English India Clays (EICL), Clariant, Ashapura Minehem Ltd., Shree Ram Minerals etc. The porous clay particle incorporated/ coated with the cationic polymer is then further coated with a hydrophobic material selected from wax or petrolatum, preferably petrolatum. The hydrophobic material is included in 0.1 to 10% by weight of the composite particle. The composite particle is included in 1 to 25%, preferably 2 to 10% by weight of the soap bar composition.

Yet another aspect of the present invention relates to use of a sanitizer composition comprising (a) 20 to 90 wt % of one or more C2 to C4 monohydric alcohol and (b) a cationic polymer having a charge density higher than 2.0 meq/g for reinfection prevention against germs. Preferably, the use is non-therapeutic or cosmetic.

The term “monohydric alcohols” as used herein refers to alcohols containing one hydroxyl group. The sanitizer composition preferably comprises 30 to 80 wt %, more preferably 40 to 75 wt %, most preferably 55 to 70 wt % of the C₂ to C₄ monohydric alcohols. In a preferred embodiment of the sanitizer composition, the monohydric alcohols are selected from ethanol, isopropyl alcohol and combinations thereof and further wherein the sanitizer composition comprises a combination of ethanol and isopropyl alcohol. Preferably, the sanitizer composition contains 30 to 80 wt. %, more preferably 40 to 70 wt. % and most preferably 50 to 68 wt. % ethanol. In a preferred embodiment the sanitizer composition comprises 2 to 15 wt. % of the one or more polyols. The term “polyols” as used herein refer to alcohols containing multiple hydroxyl groups. In a more preferred embodiment, the sanitizer composition comprises 3 to 10 wt. % of the one or more polyols.

Preferred polyols in the sanitizer composition are selected from sorbitol, glycerol, polyethylene glycol, propylene glycol, butylene glycol and combinations thereof. Even more preferred polyols in the sanitizer composition are glycerol, polyethylene glycol, propylene glycol and combinations thereof. Most preferred are propylene glycol, glycerol, butylene glycol and combinations thereof. Even most preferred is propylene glycol. The sanitizer composition preferably comprises 10 to 40 wt. % of water. More preferably the composition comprises 12 to 35 wt. % of water, most preferably 25 to 30 wt. % of water.

Yet another aspect of the present invention relates to use of a liquid cleansing composition comprising (a) a cationic polymer having a charge density higher than 2.0 meq/g (b) a surfactant and (c) higher than 65 wt % water for reinfection prevention against germs.

The present invention also relates to a liquid cleansing composition comprising (a) a cationic polymer having a charge density higher than 2.0 meq/g (b) a surfactant and (c) higher than 70 wt % water. Most preferably, the liquid cleansing compositions have water higher than 80 wt %.

The liquid cleansing composition of the invention is generally to be used for personal cleansing. It is preferably a composition for cleaning of topical surfaces like hair, scalp, body, hand or face which comprises surfactants at low concentration and are mild on skin.

Preferably, the use is non-therapeutic or cosmetic. The surfactant is generally included in 4 to 18%, preferably 6 to 12% by weight of the liquid cleansing composition.

The cationic polymer as per the invention may be included as such in the liquid cleansing composition of the invention. The cationic polymer is thus preferably included in the liquid cleansing composition in from 0.05 to 10%, preferably 0.1 to 5%, more preferably 0.1 to 2% by weight of the cleansing composition.

Surfactants for inclusion in the liquid cleansing composition of the invention may preferably be of the anionic, non-ionic, cationic or amphoteric types. When the surfactant is of the anionic class, the cationic polymer is preferably encapsulated to ensure that the cationic charge is not neutralised by the anionic charge present in the composition. To ensure this the cationic polymer may be included in the liquid cleansing composition by way of the water-in-oil emulsion composition or by way of the composite particle disclosed hereinabove.

Thus, a preferred aspect of the present invention relates to a liquid cleansing composition wherein the cationic polymer is included in the composition through an water-in-oil emulsion comprising (a) an aqueous phase comprising the cationic polymer (b) a hydrophobic phase chosen from petrolatum or wax and (c) an emulsifier.

Further, another preferred aspect of the present invention relates to a liquid cleansing composition wherein the cationic polymer is included in the composition through a composite particle comprising a porous clay particle having a water holding capacity in the range of 10 to 50% by weight, coated with the cationic polymer and further coated there upon with a hydrophobic material selected from wax or petrolatum.

A useful surfactant for inclusion in the liquid cleansing composition of the invention is sodium lauryl ether sulphate (SLES). The SLES for use in the present invention generally preferably has 1 to 3 ethoxylate (EO) groups. SLES is preferably included in 3 to 8% by weight of the composition.

The other surfactant which may be included in the present invention is cocoamide monoethanol amine (CMEA). CMEA is preferably included in 1 to 3% by weight of the composition. One preferred aspect of the liquid cleansing composition of the invention relates to an aspect wherein the surfactant comprises a mixture of sodium lauryl ether sulphate (SLES) and coco amide monoethanol amine (CMEA).

Another useful surfactant for inclusion in the liquid cleansing composition of the invention is an amphoteric surfactant preferably a betaine surfactant, more preferably an alkyl amidopropyl betaine surfactant for example cocamidopropyl betaine. In a preferred embodiment, the composition comprises from 0.1 to 5 wt. %, preferably from 0.5 to 4 wt. %, more preferably from 1 to 3 wt. % of a betaine surfactant.

Surfactants of the non-ionic class for inclusion in the liquid cleansing composition of the invention are preferably of the polyoxyethylene sorbitan alkyl esters class (sold as Tween surfactants)), fatty alcohol ethoxylates (sold as Brij surfactants), alkyl phenol ethoxylates (sold as Triton surfactants), fatty acid ethoxylates (sold as Myrj surfactants), and Alkyl poly glucosides (sold as Plantacare surfactants).

Surfactants of the cationic class which may be included in the liquid cleansing composition of the invention are cetyltrimethylammonium chloride (CTAC), cetyltrimethylammonium bromide (CTAB), or dodecyltrimethylammonium chloride (DTAC).

Water is a preferred carrier in liquid cleansing compositions of the invention. In such compositions, water is generally present in 65 to 95% by weight.

Electrolytes are preferably included in the liquid cleansing composition of the invention. The preferred electrolyte for inclusion the composition of the invention are sodium chloride, potassium chloride, sodium sulphate, sodium citrate or combinations thereof preferably sodium chloride. Electrolyte is preferably included in 0.1 to 3%, more preferably 0.1 to 2.0% by weight of the composition.

Preferred liquid cleansing compositions may include other known ingredients such as perfumes, pigments, preservatives, emollients, sunscreens, emulsifiers, gelling agents and thickening agents.

### Application on Inanimate Surfaces

The present invention relating to use of cationic polymers satisfying the charge density criterion may also be used in products for use in cleaning inanimate surfaces. Such products are known as “detergent compositions” which refers to a composition with detersive effect for treating textile article, dishware, hard surfaces and any other surfaces in the area of fabric and home care, including hard surface cleaners and/or floor and bathroom cleaners (e.g., toilet bowl cleaners). In one embodiment, the detergent composition of the present invention is a laundry detergent composition, which can be in liquid, powder, paste, gel, unit dose, pouch, or tablet form. The term “laundry detergent composition” refers to a composition that can be used to remove or to aid the removal of stains from fabric. Laundry detergent composition includes all-purpose or “heavy-duty” washing agents for fabric, as well as cleaning auxiliaries such as bleach, rinse aids, additives or pre-treat types. As used herein, the term “laundry” includes fabric surfaces, e.g., knit, woven, and non-woven surfaces.

The various forms in which detergent composition for cleaning fabric or hard inanimate surfaces are described below.

Solid detergent composition refers to a cleaning composition in the form of a solid such as a powder, a particle, agglomerate, a flake, a granule, a pellet, a tablet, a lozenge, a puck, a briquette, a brick, a solid block, a unit dose, or another solid form. Solid laundry detergent composition may be prepared by spray drying method or extrusion method or by dry mixing of the ingredients in a blender.

Bars are one preferred forms of solid detergent compositions which are also termed as “shaped” solid compositions. They are formed from moulds or extruded to possess a definite and reproducible exterior appearance. The solid shaped detergent product is preferably a bar or a cake, or in the case of laundry application it may be a tablet and is suitable for use in manual washing and/or with automatic washing machines. Processes for forming solid shaped laundry detergent compositions are well-known. These could be a soap-based composition or a composition based on non-soap surfactants like linear alkyl benzene sulphonates, or a combination of the two.

Liquid detergent composition refers to compositions which are useful for the cleaning of fabrics as well as floors, toilets, furniture and other solid surfaces at homes or in institutional locations like offices, factories, shops etc. Such liquid detergent compositions are sold in bottles usually made of plastic and are of a viscosity such that they are pourable or squeezable at room temperature.

Detergent compositions usually contain ingredients like surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzymes, enzyme inhibitors or stabilizers, enzyme activators, antioxidants, and solubilizers. The detergent composition may comprise of one or more of any type of detergent component.

### Application Using a Spray Device

Yet another aspect of the present invention relates to a hand-operable spray bottle containing a liquid cleansing composition comprising (a) a cationic polymer having a charge density higher than 2.0 (b) a surfactant and (c) higher than 80 wt % water.

In yet another preferred aspect, the present invention provides for a hand-held spray device comprising a reservoir containing the cleansing composition comprising (a) a cationic polymer having a charge density higher than 2.0 (b) a surfactant and (c) higher than 80 wt % water, said device is manually operable to produce a spray of said composition in the form of a fine aerosol.

The hand-operable spray bottle or the reservoir in the device contains the liquid cleansing composition of the present invention comprising the cationic polymer. In such products, spray bottles are typically used to direct a spray of the composition onto a surface or into the air in order to provide the benefits of the present invention. Such products may be made available in trigger-operated “gun” type bottles to spray the composition on the desired surface.

As used herein “aerosol” means a colloid of liquid droplets suspended in air. An aerosol may be produced using a propellant or without a propellant. While the spray may be generated with a propellant, it is often perceived that the presence of the propellant is considered bad for the environment and hence may be less popular with the consumers. Alternately the spray may be produced without the use of a propellant but through the use of a low boiling alcohol e.g. ethanol or isopropyl alcohol with or without water as the other diluent as the liquid cleansing composition.

The spray device is more preferably adapted for effective use by the provision of a spray mechanism having a discharge orifice which is configured to produce a fine aerosol spray having a comparatively large cone angle, suitably in the range of 55 to 80 degrees. The spray preferably has a cone angle of at least 50 degrees, preferably at least 55 degrees, more preferably at least 60 degrees. Suitably, the spray has a cone angle of no more than 90 degrees, preferably no more than 85 degrees, more preferably no more than 80 degrees. Suitable cone angles are selected from the range of 50 to 90 degrees, suitably 55 to 80 degrees, suitably 60 to 80 degrees. Preferably, the spray device is adapted for more effective use by the provision of a spray mechanism which is configured to produce a fine aerosol having a small average droplet size, suitably in the range of 20 to 200 μm. The device has a spray-direction which is substantially orthogonal to the longitudinal axis of the reservoir. Conveniently, the spray mechanism may comprise a nozzle having a discharge orifice which provides the above mentioned range of cone angle and droplet size. Advantageously, said discharge orifice may be configured to produce said fine aerosol in a substantially circular spray pattern.

The liquid cleansing composition is provided in the form of a liquid with a viscosity, such that the fine aerosol produced will suitably take the form of a fine mist. The spray mechanism may comprise a hand-operable pump. Optionally, the pump is one of a positive displacement pump; a self-priming pump; or a reciprocating pump.

The spray mechanism is preferably operated by an actuator. The actuator can be a push actuator or a pull actuator. It is envisaged that in some embodiments the reservoir will be moulded from a suitable plastic material of a type known for use in the spray device of the present invention. However, it is also possible for the reservoir to be formed from, or at least to comprise, glass or metal.

The pump is mechanically connected to an actuator e.g. a push-button for convenient actuation by a user's finger whilst holding the product. The push-button is generally mounted to the end of a plunger which extends into an internal housing of the pump and which actuates the pump when driven downwardly via operation of the push-button. An inlet of the pump is connected to an inlet pipe which generally takes the form of a length of flexible tubing. The inlet pipe facilitates the draw-up of the liquid cleansing composition from the reservoir upon operation of the spray mechanism.

The spray mechanism also comprises a nozzle which is fluidly connected to an outlet of the pump, to terminate at a discharge end. A small discharge orifice is formed in the nozzle and is configured to direct an aerosol in the form of a fine mist of the composition outwardly through the outlet aperture in the spray direction, upon operation of the spray mechanism.

In order to ensure the creation of an appropriately fine mist of the composition, the spray mechanism may comprise an atomiser. The atomiser is configured to break up a dose of the liquid drawn through the inlet tube, into a large number of small droplets and will thereby create the desired fine mist of the composition for discharge from the product. The atomiser may comprise a swirl chamber and/or a lateral dispersion chamber.

Any of the liquid cleansing compositions disclosed herein may be used in the bottle or reservoir of the spray device.

### Application Using a Towelette/Wipe

Yet another aspect of the present invention relates to a towelette product which includes:
(i) a water-insoluble substrate;
(ii) a concentrate impregnated into the substrate comprising (a) a cationic polymer having a charge density higher than 2.0 meq/g and (b) 50 to 99% by weight of the concentrate water.

By a “water insoluble substrate” is meant that the substrate does not dissolve in or readily break-apart upon immersion in water. The towelette product of the present invention is convenient to carry and use on surfaces which are difficult to wash with a cleansing composition and/or where availability of water for rinsing is scarce or inconvenient to use. Towelette products are also known as wipes.

A wide variety of materials may be used as the substrate. Nonlimiting examples of suitable substrates include nonwoven substrates, woven substrates, hydroentangled substrates, air entangled substrates and the like. Preferred embodiments employ nonwoven substrates since they are economical and readily available in a variety of materials. By “nonwoven” is meant that the layer is comprised of fibers which are not woven into a fabric but rather are formed into a sheet, particularly a tissue.

Nonwoven substrates may be comprised of a variety of materials both natural and synthetic origin. By natural is meant that the materials are derived from plants, animals, insects or byproducts. By synthetic is meant that the materials are obtained primarily from various man-made materials or from material that is usually a fibrous web comprising any of the common synthetic or natural textile-length fibers, or mixtures thereof.

Nonlimiting examples of natural materials useful in the present invention are silk fibers, keratin fibers and cellulosic fibers. Nonlimiting examples of keratin fibers include those selected from the group comprising wool fibers, camel hair fibers, and the like. Nonlimiting examples of cellulosic fibers which are preferred for use as a water-insoluble substrate in the towelette product of the present invention include those selected from the group comprising wood pulp fibers, cotton fibers, hemp fibers, jute fibers, flax fibers, and mixtures thereof.

Nonlimiting examples of synthetic materials useful in the present invention include those selected from the group comprising acetate fibers, acrylic fibers, cellulose ester fibers, modacrylic fibers, polyamide fibers, polyester fibers, polyolefin fibers, polyvinyl alcohol fibers, rayon fibers and mixtures thereof.

For the purposes of the present invention the most preferred towelettes comprise non-woven substrates. The substrate can be made into a wide variety of shapes and forms.

Generally, the substrate is in single use towelette form. Advantageously, the towelettes are folded in a Z-shaped formation. They may be interleaved with one another but preferably are not interleaved. Advantageously the size of the towelette may range in length from 10 to 40 cm, preferably from 15 to 30 cm, optimally from 18 to 24 cm. The width of the towelette may range from 8 to 30 cm, preferably from 10 to 25 cm, optimally from 15 to 20 cm.

Anywhere from 5 to 100, preferably from 10 to 50 single towelettes may be stored within a dispensing pouch, preferably a moisture impermeable pouch. During storage and between dispensing, the pouch is resealable, usually via an adhesive strip covering a dispensing opening. Single towelette containing may be employed. Single towelette pouches also

The amount of concentrate relative to the substrate may range from about 20:1 to 1:20, preferably from 10:1 to about 1:10 and optimally from about 2:1 to about 1:2 by weight.

The concentrate impregnated into the substrate may additionally comprise a surfactant. Surfactants as disclosed herein for use in liquid cleansing compositions may be used. The concentrate may additionally comprise emollients which are deposited on the surface along with the other actives. These emollients include, for example, esters, hydrocarbons or dimethicone oils. The presence of the emollients obscures any stickiness which may result from evaporation of water or the carrier leaving dried active behind.

Typically, a humectant is incorporated in the concentrate impregnated in the towelette of the present invention. Humectants are normally polyols. Representative polyols include glycerin, diglycerin, polyalkylene glycols and more preferably alkylene polyols and their derivatives including propylene glycol, dipropylene glycol, polypropylene glycol, polyethylene glycol and derivatives thereof, sorbitol, hydroxypropyl sorbitol, hexylene glycol, 1,2butylene glycol, 1,2,6-hexanetriol, isoprene glycol, ethoxylated glycerol, propoxylated glycerol and mixtures thereof. The most preferred is 2methyl-1,3-propanediol available as MP Diol from the Arco Chemical Company. Amounts of the polyol may range from about 0.5 to about 95%, preferably from about 1 to about 50%, more preferably from about 1.5 to 20%, optimally from about 3 to about 10% by weight of the concentrate.

Concentrates is usually provided with a variety of cosmetically acceptable carrier vehicles. Normally the carrier vehicle is water. Amounts of the carrier vehicle may range from about 50 to about 99%. The carrier is preferably present in as high as 95%, and in some cases as high as 97 wt % of the impregnating composition.

Preservatives can desirably be incorporated into the concentrate to protect against the growth of potentially harmful microorganisms. Suitable traditional preservatives are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate, caprylyl glycol and benzyl alcohol. Preservatives are preferably employed in amounts ranging from 0.01% to 2% by weight of the concentrate.

### Method of the Invention

The present invention also relates to a method of minimizing regrowth of germs on a surface for up to 6 hours post cleaning comprising the step of cleaning the surface with a composition comprising a cationic polymer having a charge density higher than 2.0 meq/g. The surface could be either an animate or an inanimate surface. The method is preferably effective against regrowth of germs on the topical surface of a human body. The method is preferably for use in non-therapeutic or cosmetic applications.

Thus, the present invention relates to use of a cationic polymer having a charge density higher than 2.0 meq/g for reinfection prevention against germs.

Preferably, the present invention further relates to use of a cationic polymer having a charge density higher than 2.0 meq/g further having a nitrogen content of higher than 5% by weight of the polymer for reinfection prevention against germs.

Preferably, the present invention relates to use of a cationic polymer having a charge density higher than 3.5 meq/g, further having a nitrogen content of higher than 5% by weight of the polymer for reinfection prevention against germs. Preferably the polymer is PDADMAC.

The present invention also relates to use of a water-in-oil emulsion composition comprising (a) an aqueous phase comprising a cationic polymer having a charge density higher than 2.0 meq/g (b) a hydrophobic phase chosen from petrolatum or wax and (c) an emulsifier, for reinfection prevention against germs. Preferably, the water-in-oil emulsion composition additionally comprises an anionic surfactant, preferably soap. Preferably, the water-in-oil emulsion is a bar.

The present invention also relates to use of a soap bar composition comprising (a) 40 to 80 wt % soap and (b) a composite particle comprising a porous clay particle having a water holding capacity in the range of 10 to 50% by weight, coated with a cationic polymer having a charge density higher than 2.0 meq/g, and further coated there upon with a hydrophobic material selected from wax or petrolatum, for reinfection prevention against germs.

The present invention also relates to use of a sanitizer composition comprising (a) 20 to 90 wt % of one or more C₂ to C₄ monohydric alcohol and (b) a cationic polymer having a charge density higher than 2.0 meq/g, for reinfection prevention against germs.

The present invention also relates to use of a liquid cleansing composition comprising (a) a cationic polymer having a charge density higher than 2.0 meq/g (b) a surfactant and (c) higher than 65% water, for reinfection prevention against germs.

The invention will now be illustrated with the help of the following non-limiting examples.

### EXAMPLES

### Examples A-C, 1: Antiviral Efficacy of PDADMAC Deposited on Vitro-Skin Against Adenovirus

The following samples as given below were tested against Adenovirus using the protocol as given thereafter:
Example A: Control sample
Example B: Adenovirus on Vitro skin
Example C: Vitroskin treated with a commercially available soap bar sample
Example 1: Vitroskin treated with a soap bar sample containing 4 wt % water-in-oil emulsion containing 1% PDADMAC by weight of the soap bar.

The water-in-oil emulsion and the soap bar containing it were prepared as follows:

The water-in-oil emulsion (as in Table-1 below) was first prepared.

TABLE 1

| Ingredient | Wt % |
|---|---|
| Petrolatum | 32 |
| Sorbitan Monooleate (SMO) | 2 |
| Sorbitan Monostearate (SMS) | 1.5 |
| PDADMAC (Merquat 100) | 25 |
| Water | To 100 |

Emulsion making process:
1. Dose the pre-weighed quantity of petrolatum in mixing vessel and heat the mass till 82° C.
2. Put this mass under continuous stirring and add the emulsifiers (SMO and SMS) till the temperature is stabilized to 82° C.
3. Measure required quantity of Merquat-100 and DM (demineralised) water in another vessel and heat the mass till 82° C. under continuous stirring. It is useful to have dispersed and continuous phase at similar temperatures above 80° C. to avoid cooling of emulsion mass when mixed.
4. Put the petrolatum with emulsifier mass (Oil phase) under Silverson homogenizer and set the Silverson at 2000 RPM. Dose the Merquat-100 with water (Water phase) in the mixing vessel at the rate of 70 ml/min and gradually increase the Silverson speed to 5000 RPM.
5. Continue the mixing for 20 minutes
6. After emulsification, cool the mass under ambient setting till 30° C.
7. Check the viscosity at 30° C. with LV4 spindle, 6 rpm. It is preferably higher than 125000 cps.
8. Transfer the material in storage jar for further use.

The above water in oil emulsion was used to prepare a soap bar as shown in Table-2 by the conventional milled and plodded route.

TABLE 2

| | |
|---|---|
| Anhydrous Soap | 70 |
| Glycerin | 2 |
| Talc | 4 |
| PDADMAC Emulsion* | 4 |
| Fragrance | 1 |
| Sodium Chloride | 0.8 |
| Water and minors | Up to 100 |

*as shown in table 1

The samples of Examples A-C and Example -1 were used to test the reinfection prevention efficacy against the adenovirus using the following protocol:

### Propagation of Adenoviruses

Hela cell line (ATCC, Catalogue no. CCL-2) was used to propagate and titer Adenoviruses (ATCC, Catalogueno. VR-5). Hela cells were cultured in 10% DMEM growth medium at 37° C. in a tissue culture incubator containing 5% carbon dioxide (CO₂). During infection, the virus seed stock was diluted in virus growth medium (DMEM+2% FCS) and added to a confluent monolayer of Hela cells at a multiplicity of infection (M.O.I.) of 0.1 in T-75 flask and left to adsorb for 1 hour at 37° C. The flask was rocked every 10 minutes. After 1 hour, 10 ml of virus growth medium was added to each flask. The flask was incubated at 37° C. and 5% CO₂ for 5 days and virus harvested when 90% of the monolayer showed virus-specific cytopathic effects (CPE). The cultures were freeze-thawed three times and centrifuged at 3000 g for 10 min at 4° C. to remove cell debris. Aliquots of supernatant (300 μl) were made and stored at −70° C.

### Viral titration

Monolayers of Hela cells (for adenovirus) and MDCK cells (ATCC, catalogue no. CCL-34) were grown in 96-well tissue-culture plates (Costar) at 37° C. Tenfold dilution of virus suspensions were prepared in virus growth medium. One hundred micro litres of each dilution were added to five replicate wells in 96-well tissue-culture plates. Cell controls were included in each plate. Plates were incubated at 37° C. and observed daily for virus induced cytopathic effect for a period of four to five days. The virus titre was calculated by TCID50 (Tissue culture infective dose) using the Spearmen-Karber formula.

### Determination of Virucidal Activity of Active Deposited From Soap Post-Wash on Vitro-Skin

5×5 cm² section of VITRO-SKIN (IMS Inc., catalogue no. N-19_20 Sheets) was placed in a petri dish. VITRO-SKIN was washed by pipetting 2 ml of sterile DI water onto the skin and gently shaking covered petri dish in a back and forth motion till water covers the whole surface area of the vitro-skin. The covered petri dish is then set down for 10 minutes. After 10 minutes, water was removed, and wet vitro-skin was transferred with forceps to a new petri-dish to dry for 20 minutes. During drying, the lid of the petri dish would be half on, half off the dish.

After drying, the skin was treated with the test product/water, as follows.

Before application, test products (half-cut soap bars) were wetted for 10 seconds. Wet half-cut bars were be applied to the VITRO-SKIN by rubbing wet half-cut bar against the test area in a horizontal pattern for 15 seconds (petri-plate should rest on the surface of the biosafety hood) After 15 seconds, 1 ml of water was applied to the VITRO-SKIN with a pipette and spread using an L-spreader to cover the test area for 45 seconds in horizontal and vertical pattern (this would lead to lathering of the soap) (petri-plate should rest on the surface of the biosafety hood) The VITRO-SKIN was then held vertically from one corner using forceps and rinsed by pouring 20 ml water down the skin and into a petri dish below.

After application of the test product/water, the VITRO-SKIN was dried by placing the skin over the lid of a petri dish with the lid half on, half off the dish. The skin was allowed to dry for 30 minutes.

After drying, the skin was transferred to a petri dish with 1% agarose (treated side facing upward). The test area was then inoculated with 100 μl of the viral suspension.

The suspension was spread over the test area using an L-spreader until completely absorbed (approximately 2 minutes). The petri dish was then incubated at 37° C. for 6 hours (±5 minutes).

During incubation, the microspin column was set for each sample (S-200, GE Healthcare) by vortexing and removing the tip of the column. The column was then centrifuged for 1 minute at 4° C., 735 g. After centrifugation, the column was kept at 4° C. until use.

After the 6-hour incubation, the VITRO-SKIN was placed into 9 ml of neutralizing solution (D/E neutralizer) in a 50 ml conical tube and vortexed for 5 minutes straight. After neutralization, 100 μl of neutralized virus was added to the prepared column and eluted by centrifuging for 2 min at 4° C., 735 g. The surviving viral load was then titrated via standard TCID50 assay.

Measurement of TCID₅₀: To determine TCID50, 0.1 ml of each dilution was added into five wells of a microtiter plate (96 well plate) containing a confluent (>70%) cell monolayer without any medium. The plates were incubated at 37° C. (+/−1° C.) at 5% CO₂ for the relevant time-period. The cytopathic effect was observed and TCID50 was calculated using Spearman-Kärber method.

### TCID₅₀ Determination by the Spearman-Karber Method:

The Spearman-Karber method is very simple and gives results most compatible with other calculations. Prerequisite of the evaluation is the use of several dilutions which cover infection of all cell culture units to those in which no virus multiply. The mean and standard deviation can only be calculated; when several test results are available.

The formula is:

Negative logarithm of the 50% end point=Negative logarithm of the highest virus concentration used−[(Sum of % affected at each dilution/100−0.5)×(log of dilutions)]

| Dilution | Result ^{a} | % positive |
|---|---|---|
| 4 | 444444 | 100 |
| 5 | 444344 | 100 |
| 6 | 443003 | 66.7 |
| 7 | 400020 | 33.3 |
| 8 | 000000 | 0 |

| | |
|---|---|
| Sum of % positive | 300 |

^{a} 1 to 4: virus present, degree of CPE in 6 cell culture units
0: no virus present

Using the data from above table the 50% end point is calculated as follows:

Log₁₀TCID₅₀=−4−[{(100+100+66.7+33.3+0)/100}−0.5]×1=−4−[300/100−0.5]×1=−4−2.5×1=−6.5

Therefore, the 50% end point is 10^{-6.5} or Log₁₀ TCID₅₀=6.5

The data on reinfection prevention of adenovirus of the various samples Examples A-C and Example-1 is summarized in Table-3 below:

TABLE 3

| Example | Sample | Log TCID 50/0.1 ml |
|---|---|---|
| A | Control | 4.5 |
| B | Adenovirus on vitroskin | 4.4 |
| C | Commercial soap | 4.4 |
| 1 | Soap bar with PDADMAC | 2.2 |
| | (as in table 2 above) | |

The data in Table-3 above indicates that soap bar containing PDADMAC (as per the invention) is able to inhibit infection by virus for six hours after treatment with the cationic polymer.

### Example D-F. 2: Antiviral Efficacy of PDADMAC Deposited on Vitro-Skin Against Influenza Virus

The samples as shown below in Table-4 were tested against Influenza virus (ATCC catalogue no. VR-1469) using a procedure similar to that conducted for Adenovirus, except that the propagation of influenza virus was as given below:

### Propagation of Influenza Viruses

MDCK cell line was used to propagate and titer Influenza viruses.

MDCK cells were cultured in growth medium (DMEM+10% FCS+0.1% PS) at 37° C. in a tissue culture incubator containing 5% carbon dioxide (CO₂). Before adding viruses to the confluent layer of MDCK cells, cells were washed with media containing TPCK treated trypsin. During infection, the virus seed stock was diluted in virus growth medium (maintenance medium+0.0001% TPCK treated trypsin) and added to the confluent monolayer of MDCK cells at a multiplicity of infection (M.O.I.) of 0.1 in T-75 flask and left to adsorb for 1 hour at 37° C. The flask was rocked every 10 minutes. After 1 hour, 10 ml of virus growth medium was added to each flask. The flask was incubated at 37° C. and 5% CO₂ for 5 days and virus harvested when 90% of the monolayer showed virus-specific cytopathic effects (CPE). The cultures were freeze-thawed three times and centrifuged at 3000 g for 10 min at 4° C. to remove cell debris. Aliquots of supernatant (300 μl) were made and stored at −70° C.

The data is summarised in Table-4.

TABLE 4

| Example | Sample | Log TCID 50/0.1 ml |
|---|---|---|
| D | Control | 4.4 |
| E | Influenza on vitroskin | 3.2 |
| F | Commercial soap | 3.0 |
| 1 | Soap bar with PDADMAC | 1.6 |
| | (as in table 2 above) | |

The data in Table-4 indicates that results similar to that with adenovirus can be obtained with influenza virus for reinfection prevention using the cationic polymer claimed in the present invention.

### Examples G, H, 3: Effect of Cationic Charge Density of Polymers

The effect of charge density of polymers was studied in neat actives as shown in Table-5 below. The procedure used was the same as described earlier but the samples used were neat at the concentration indicated in table-5 (and not through soap bars as described in previous examples).

TABLE 5

| Example | Sample | Log TCID 50/0.1 ml |
|---|---|---|
| G | Adenovirus on vitroskin | 4.5 |
| H | 0.1% cationic guar gum Jaguar* | 4.5 |
| | with charge density of 0.9 meq/g | |
| 3 | 0.1% PDADMAC with charge | 2.6 |
| | density of 6.2 meq/g | |

*(From: Solvay, Jaguar ® C-17)

The data in the above table indicates that high charge density polymer is essential to get the benefits of the present invention.

### Example-4: Kinetics of Post-Wash Adenoviral Inactivation by PDADMAC on Vitro Skin

The protocol used to measure the adenoviral inactivation over various time points is the same as that which has been described previously under the subheading: Determination of virucidal activity of active deposited from soap post-wash on vitro-skin. The only difference lies in the duration after which the virus is harvested. Instead of single time-point as indicated earlier, harvesting of virus was performed at each time-point as referred to below in table-6.

The log reduction of adenovirus over the time frame of 5 minutes to 5 hours as measured using the above protocol is summarized in the table-6 below:

TABLE 6

| | |
|---|---|
| Time | Log reduction |

| | | |
|---|---|---|
| 5 | mins | 1.0 |
| 15 | mins | 1.0 |
| 30 | min | 1.0 |
| 1 | hour | 1.0 |
| 2 | hours | 1.0 |
| 3 | hours | 1.0 |
| 4 | hours | 2.0 |
| 5 | hours | 2.0 |

The data in Table-6 above indicates that the minimum time required for PDADMAC to inactivate virus was within 5 minutes. 1 log reduction in the adenovirus titre was observed after 5 mins of wash by PDADMAC emulsion bar (of Example-1) on vitro-skin and 2 log reduction in the virus titre was observed minimum after 4 hours of wash by the PDADMAC emulsion bar.

### Examples 5-6: Liquid Cleansing Compositions as Per the Invention

The following liquid cleansing composition (in Table-7 and Table-8) as per the invention give the desired beneft of reinfection prevention against virus over long period of time.

TABLE 7
(Example - 5)

| Ingredient | Wt % |
|---|---|
| SLES. 1EO (70% in water) | 7.1 |
| CMEA | 2.5 |
| PDADMAC water-in-oil emulsion (as prepared for Example -1) | 1.0 |
| Sodium benzoate | 0.5 |
| Perfume | 0.5 |
| Sodium chloride | 0.35 |
| Citric acid monohydrate | 0.2 |
| Tetra sodium EDTA (39% pure) | 0.13 |
| Water | To 100 |

TABLE 8
(Example -6)

| Ingredient | Wt % |
|---|---|
| SLES. 1EO (70% in water) | 9.0 |
| CAPB (30% in water) | 5.0 |
| PDADMAC water-in-oil emulsion (as prepared for Example -1) | 1.0 |
| Sodium benzoate | 0.5 |
| Perfume | 1.0 |
| Sodium chloride | 1.0 |
| Citric acid monohydrate | 0.4 |
| Tetra sodium EDTA (39% pure) | 0.13 |
| Water | To 100 |

### Examples I,J, 7-9: Antiviral Efficacy of PDADMAC on Vitro-Skin Against Adenovirus Delivered Through a Sanitizer Composition

A sanitizer composition as shown in Table-9 below was prepared.

TABLE 9

| Ingredients | wt % |
|---|---|
| Ethyl alcohol | 62 |
| Isopropyl alcohol | 3 |
| Water | 3 |
| Niacinamide | 1 |
| Propyleneglycol | 5 |
| Ag DTPA | 0.0067 |
| Merquat 100 (PDADMAC) | As in Table - 10 |
| Hydoxypropyl methyl cellulose (as Benecel E10 M) | 1.5 |
| Diisopropyl adipate (as Ceraphyl 230) | 5 |
| Perfume | 0.2 |
| Water | To 100 |

The compositions as shown in Table-10 below were measured for reinfection prevention against adenovirus (N=2) and represented as Log TCID₅₀ titre.

Fixed amount of the composition with the three different PDADMAC concentrations was applied on vitro-skin and allowed to dry for intervals of 6 hr. Post drying, Adenovirus was added on vitro-skin and incubated for 5 mins contact time. Post addition, Adenovirus was harvested and added to target cells and incubated for Cytopathic effects (CPE). The CPE is represented as Log TCID₅₀ titre. The data is summarized in Table-10 below.

TABLE 10

| Example | Ingredient | Log TCID50 |
|---|---|---|
| I | Control Adenovirus | 5.00 |
| J | Sanitizer base* | 4.70 |
| 7 | Sanitizer base* + 0.25% PDADMAC | 4.00 |
| 8 | Sanitizer base* + 0.50% PDADMAC | 3.60 |
| 9 | Sanitizer base* + 1.0% PDADMAC | 3.00 |

*As shown in table 9

The data in the table-10 above indicates that the benefits of reinfection prevention on skin against virus can be delivered through a sanitizer composition comprising the specific cationic polymer.

### Examples K,L, 10, 11: Antibacterial Efficacy in the Form of Reinfection Prevention Through Use of PDADMAC on Vitro-Skin Against E. coli Delivered Through a Sanitizer Composition

A sanitizer composition as shown in Table-9 above was prepared. From it, two compositions were prepared at 0.5% and 1.0% PDADMAC. The compositions were tested for prevention of reinfection against E. coli (ATCC 10536) using the following procedure:

Fixed amount of the test product was added onto the VITRO-SKIN®. After application of the test product, the petri plates were incubated at a 37±1° C. The untreated VITRO-SKIN® controls were processed in the same manner. After incubation period of 3 hours (±5minutes) of the treated VITRO-SKIN®, the test area was inoculated with the bacterial suspension. The suspension was spread uniformly and after 5 min, the VITRO-SKIN® was transferred aseptically to neutralizer tubes. The residual bacteria was dislodged from the VITRO-SKIN®.

The Residual bacteria was enumerated by standard microbiological technique and was represented as Log CFU/cm². The data on the various samples is summarized in Table-11.

TABLE 11

| | | Log CFU/cm² |
|---|---|---|
| Example | Ingredient | (E. coli 10536) |
| K | Control culture | 6.27 |
| L | Sanitizer base* | 6.31 |
| 10 | Sanitizer base* + 0.5% PDADMAC | 2.32 |
| 11 | Sanitizer base* + 1% PDADMAC | 2.02 |

*As shown in table 9

The data in the table-11 above indicates that the benefits of reinfection prevention on skin is also obtained against a bacteria like E. coli and that this can be delivered through a sanitizer composition comprising the specific cationic polymer.

### Examples M-O, 13-14: Antibacterial Efficacy in the Form of Reinfection Prevention Through Use of Various Cationic Polymers on Vitro-Skin Against S. aureus Delivered Through a Sanitizer Composition

Sanitizer compositions as shown in Table-12 below was prepared. The compositions were tested for prevention of reinfection against E. coli (ATCC 10536) using the following procedure:

TABLE 12

| Ingredients | wt % |
|---|---|
| Ethyl alcohol | 62 |
| Isopropyl alcohol | 3 |
| Cationic polymer | As in Table - 13 |
| Hydoxypropyl methyl cellulose (as Benecel E10 M) | 1.5 |
| Water | To 100 |

Fixed amount of the test product was added onto the VITRO-SKIN®. After application of the test product, the petri plates were incubated at a 37±1° C. The untreated VITRO-SKIN® controls were processed in the same manner. After incubation period of 1 hour of the treated VITRO-SKIN®, the test area was inoculated with the bacterial suspension. The suspension was spread uniformly and after 5 min, the VITRO-SKIN® was transferred aseptically to neutralizer tubes. The residual bacteria was dislodged from the VITRO-SKIN®. The Residual bacteria was enumerated by standard microbiological technique and was represented as Log CFU/cm². The data on the various samples is summarized in Table-13.

TABLE 13

| | | Charge density of | Log CFU/cm² |
|---|---|---|---|
| Example | Ingredient | Cationic polymer | (S. aureus 6538) |
| M | Control culture | — | 5.46 |
| | (untreated skin) | | |
| N | Sanitiser base* | — | 5.62 |
| | (without polymer) | | |
| O | Sanitiser base* + | 1.25 | 5.52 |
| | 1% PQ-10 | | |
| 12 | Sanitiser base* + | 2.3 | 2.23 |
| | 1% PQ-37 | | |
| 13 | Sanitiser base* + | 3.3 | 3.94 |
| | 1% PQ-16 | | |
| 14 | Sanitiser base* + | 6.2 | 1.75 |
| | 1% MQ-100 | | |

*As shown in table 12

The data in the table-13 above indicates that the benefits of reinfection prevention on skin is also obtained against a bacteria like S. aureus and that this can be delivered through a composition comprising the cationic polymers having a charge density higher than 2.0 meq/g.

1. A cationic polymer having a charge density higher than 2.0 meq/g for use inconfigured for preventing reinfection against germs.

2. The cationic polymer as claimed in claim 1 having a nitrogen content of higher than 5% by weight of the polymer for use inconfigured for preventing reinfection against germs.

3. The cationic polymer as claimed in claim 1 having a charge density higher than 3.5 meq/g.

4. A water-in-oil emulsion composition comprising (a) an aqueous phase comprising a cationic polymer as claimed in claim 1, having a charge density higher than 2.0 meq/g, (b) a hydrophobic phase chosen from petrolatum or wax; and (c) an emulsifier, inconfigured for preventing reinfection against germs.

5. The water-in-oil emulsion composition as claimed in claim 4, additionally comprising an anionic surfactant configured for preventing reinfection against germs.

6. The water-in-oil emulsion composition as claimed in claim 5. wherein the composition is a bar.

7. A soap bar composition comprising (a) 40 to 80 wt % soap and (b) a composite particle comprising a porous clay particle having a water holding capacity in the range of 10 to 50% by weight, coated with a cationic polymer as claimed in claim 1, having a charge density higher than 2.0 meq/g, and further coated there upon with a hydrophobic material selected from wax or petrolatum, configured for preventing reinfection against germs.

8. A sanitizer composition comprising (a) 20 to 90 wt % of one or more C₂ to C₄ monohydric alcohol and (b) a cationic polymer as claimed in claim 1, having a charge density higher than 2.0 meq/g, configured for preventing reinfection against germs.

9. A liquid cleansing composition comprising (a) a cationic polymer as claimed in claim 1, having a charge density higher than 2.0meq/g (b) a surfactant and (c) higher than 65% by weight water, for use inconfigured for preventing reinfection against germs.

10. (canceled)

11. The liquid cleansing composition as claimed in claim 9, wherein the cationic polymer is included in the composition through a water-in-oil emulsion comprising (a) an aqueous phase comprising the cationic polymer (b) a hydrophobic phase chosen from petrolatum or wax and (c) an emulsifier.

12. The liquid cleansing composition as claimed in claim 9, wherein the cationic polymer is included in the composition through a composite particle comprising a porous clay particle having a water holding capacity in the range of 10 to 50% by weight, coated with the cationic polymer, and further coated there upon with a hydrophobic material selected from wax or petrolatum.

13. A hand-held spray device comprising a reservoir containing a liquid cleansing composition as claimed in claim 9, comprising (a) a cationic polymer having a charge density higher than 2.0 (b) a surfactant and (c) higher than 65% water; said device is manually operable to produce a spray of said composition in the form of a fine aerosol.

14. A towelette product which includes:
(i) a water-insoluble substrate;
(ii) a concentrate impregnated into the substrate comprising (a) a cationic polymer as claimed in claim 1, having a charge density higher than 2.0 meq/g and (b) 50 to 99% by weight of the concentrate water.

15. A method of minimizing regrowth of germs on a surface for up to 6 hours post cleaning comprising the step of cleaning the surface with a composition comprising a cationic polymer as claimed in claim 1, having a charge density higher than 2.0 meq/g.

16. The cationic polymer as claimed in claim 1, wherein the cationic polymer is polydiallyldimethylammonium chloride.

17. The water-in-oil emulsion composition as claimed in 5, wherein the anionic surfactant is soap.
